(19) United States
(12) Patent Application Publication
(10) Pub. No.: US 2009/0221984 A1
(43) Pub. Date: Sep. 3, 2009

(54) Method and apparatus for providing therapeutically effective dosage formulations of lidocaine with and without epinephrine

(76) Inventors: Akram Girgis, Deerfield Beach, FL (US); Steve Thuss, Wellington, FL (US)

Correspondence Address: Mitchell A. Stein, Esq.;STEIN LAW, P.C., 24 Woodbine Avenue, Suite 4, Northport, NY, 11768, US

(21) Appl. No.: 12/074,296

(22) Filed: Mar. 3, 2008

(51) Int. Cl. A61B 19/00; A61K 31/167; B65D 25/08

(52) U.S. Cl. 604/416; 514/626; 206/219

(57) ABSTRACT

An apparatus for providing therapeutically effective mixtures of buffered lidocaine (with and without epinephrine) while simultaneously increasing the shelf life, having a two-chambered medicine vial, in which one chamber isolates up to 20 ml of a lidocaine solution (1% with or without epinephrine) and the other chamber isolates up to 2 ml of sodium bicarbonate (8.4%), with a fracturable wall therebetween such that the wall is fractured by bending or twisting, for mixing the lidocaine and sodium bicarbonate to create a buffered lidocaine for administration in therapeutically effective amounts, and further comprising stopper means, like a rubber stopper, for the insertion of a syringe for extraction of the mixture for administration. Also shown is a method for isolating the components for shelf life and providing mixing for an effective composition.

### FIELD OF THE INVENTION

The present invention relates to the field of topical anaesthetics and more specifically to buffering a specific anaesthetic in a manner that provides efficacy while not reducing shelf life, used to improve surgical techniques by minimizing pain and bleeding, while avoiding painful side effects caused by such administration.

### BACKGROUND OF THE INVENTION

Lidocaine or lignocaine is a local anesthetic and antiarrhythmic drug most commonly marketed by AstraZeneca under the names “Xylocalne” and “Xylocard.”

By way of background, lidocaine, was the first amino amide-type local anesthetic, which was ostensibly developed by Nils Löfgren and Bengt Lundqvist in 1943 and first marketed in 1948. Lidocaine has a more rapid onset of action and longer duration of action than amino ester-type local anesthetics such as procaine. It is approximately 90% metabolized in the liver by CYP1A2 (and to a minor extent CYP3A4) to the pharmacologically-active metabolites monoethylglycinexylidide and glycinexylidide.

The elimination half-life of lidocaine is approximately 1.5-2 hours in most patients, rendering it specifically effective as a local anaesthetic as it is quickly dispatched by the body after operations are completed. (It is also known that this half-life may be prolonged in patients with specific medical conditions, like hepatic impairment (wherein the average is 343 minutes) or congestive heart failure (wherein the average is 136 minutes).

Lidocaine's chemical, two dimensional conformation, is shown below:

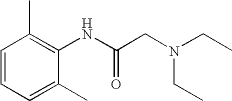

In terms of efficacy, lidocaine alters depolarization in neurons, by blocking the fast sodium (Na+) channels in the cell membrane. With sufficient blockade, the membrane generally does not depolarize and thus eliminates transmission of an action potential, leading to its anesthetic effects.

In terms of lidocaine's utility as an antiarrhythmia drug, lidocaine is classified as a Class Ib antiarrhythmic agent, blocking the sodium channel of the cardiac action potential, which reduces the slope of phase 0 of depolarization with little effect on the PR interval, QRS complex or QT interval.

Clinically, lidocaine is used for topical, infiltration, nerve block, ophthalmic, epidural and intrathecal anaesthesia, IV regional anaesthesia (IVRA). It is also used for treatment of serious ventricular arrhythmias (IV preparations), including VF (Ventricular Fibrillation) associated with cardiac arrest. Lastly, lidocaine has been employed in minimizing neuropathic pain, including postherpetic neuralgia.

Contraindications for the use of lidocaine include heart block, second or third degree (without pacemaker), severe sinoatrial block (without a pacemaker), serious adverse drug reaction to lignocaine or amide local anaesthetics, and concurrent treatment with quinidine, flecainide, disopyramide, procainamide (Class I antiarrhythmic agents).

Adverse drug reactions (ADRS) are rare when lidocaine is used as a local anesthetic and is administered correctly. Most ADRs associated with lidocaine for anesthesia relate to administration technique (resulting in systemic exposure) or pharmacological effects of anesthesia; however allergic reactions can rarely occur.

Systemic exposure to excessive quantities of lidocaine mainly result in central nervous system (CNS) and cardiovascular effects—CNS effects usually occur at lower blood plasma concentrations and additional cardiovascular effects present at higher concentrations, though cardiovascular collapse may also occur with low concentrations. CNS effects may include CNS excitation (nervousness, tingling around the mouth, tinnitus, tremor, dizziness, blurred vision, seizures) followed by depression (drowsiness, loss of consciousness, respiratory depression and apnea). Cardiovascular effects include hypotension, bradycardia, arrhythmias, and/or cardiac arrest—some of which may be due to hypoxemia secondary to respiratory depression.

ADRs associated with the use of intravenous lidocaine are similar to toxic effects from systemic exposure listed above. These are dose-related and more frequent at high infusion rates. Common ADRs include: headache, dizziness, drowsiness, confusion, visual disturbances, tinnitus, tremor, and/or paraesthesia. Infrequent ADRs associated with the use of lidocaine include: hypotension, bradycardia, arrhythmias, cardiac arrest, muscle twitching, seizures, coma, and/or respiratory depression.

Dosage forms for lidocaine are typically in the form of lidocaine hydrochloride, available in various forms, including injected as a local anesthetic (at times combined with epinephrine), transdermal patch (sometimes combined with prilocaine), intravenous injection (also at times combined with epinephrine), intravenous infusion, nasal instillation/spray (combined with phenylephrine), oral gel (often referred to as “viscous lidocaine” or abbreviated “lidocaine visc” or “lidocaine hcl visc” in pharmacology), oral liquid, and topical gel (at times combined with aloe vera gels), and as a topical liquid.

Lidocaine is generally presented in aqueous solution at 1 or 2% by volume. Since the typical form is as a hydrochloride (aqueous), it is generally known that the composition is acidic and results in a “stinging” or other painfully sensation when used as a local or IV injection. It is thus an object of the instant invention to provide a method and apparatus for buffering lidocaine to heighten its pH while maintaining its efficacy for a sufficient shelf life for medical uses.

Often in use, to increase efficacy, lidocaine solutions also include epinephrine. Epinephrine is a catecholamine, a sympathomimetic monoamine derived from the amino acids phenylalanine and tyrosine. The Latin roots ad-+renes and the Greek roots epi-+nephros both literally mean “on/to the kidney” (referring to the adrenal gland, which secretes epinephrine). Epinephrine is sometimes shortened to “epi” in medical jargon.

In May 1886, William Bates reported the discovery of a substance produced by the adrenal gland in the New York Medical Journal. Epinephrine was isolated and identified in 1895 by Napoleon Cybulski, a Polish physiologist. The discovery was repeated in 1897 by John Jacob Abel. Jokichi Takamine discovered the same hormone in 1900, without knowing about the previous discovery. It was first artificially synthesized in 1904 by Friedrich Stolz.

Epinephrine's two-dimensional conformation is shown below:

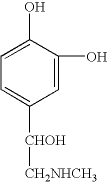

Epinephrine plays a central role in short-term stress reaction—the physiological response to threatening, exciting or environmental stressor conditions such as high noise levels or bright light. In the body, it is secreted by the adrenal medulla. When released into the bloodstream, epinephrine binds to multiple receptors and has numerous effects throughout the body. It increases heart rate and stroke volume, dilates the pupils, and constricts arterioles in the skin and gut while dilating arterioles in leg muscles. It elevates the blood sugar level by increasing catalysis of glycogen to glucose in the liver, and at the same time begins the breakdown of lipids in adipocytes. Epinephrine has a suppressive effect on the immune system.

Epinephrine is used as a drug to promote peripheral vascular resistance via alpha-stimulated vasoconstriction in cardiac arrest and other cardiac dysrhythmias resulting in diminished or absent cardiac output, such that blood is shunted to the body's core. This beneficial action comes with a significant negative consequence—increased cardiac irritability—which may lead to additional complications immediately following an otherwise successful resuscitation. Alternatives to this treatment include vasopressin, a powerful antidiuretic which also increases peripheral vascular resistance leading to blood shunting via vasoconstriction, but without the attendant increase to myocardial irritability.

As a result of its suppressive effect on the immune system, epinephrine is used to treat anaphylaxis and sepsis. Allergy patients undergoing immunotherapy may receive an epinephrine rinse before the allergen extract is administered, thus reducing the immune response to the administered allergen. It is also used as a bronchodilator for asthma if specific beta2-adrenergic receptor agonists are unavailable or ineffective. Adverse reactions to epinephrine include palpitations, tachycardia, anxiety, headache, tremor, hypertension, and acute pulmonary edema.

Epinephrine release is stimulated by the sympathetic nervous system. As the adrenal medulla is essentially a modified sympathetic ganglion, it is activated by preganglionic sympathetic fibers which secrete acetylcholine, activating nicotinic acetylcholine receptors on the adrenal medullary cells, causing them to secrete epinephrine.

Cortisol, a product of chronic sympathetic nervous system activation, also enhances epinephrine production by increasing the synthesis of phenol O-methyltransferase (POMT), the adrenal medullary enzyme responsible for the conversion of norepinephrine to epinephrine.

Epinephrin binds its receptor, that associates with an heterotrimeric G protein. The G protein associates with adenylate cyclase that converts ATP to cAMP. Epinephrin binds its receptor, that associates with an heterotrimeric G protein. The G protein associates with adenylate cyclase that converts ATP to cAMP, spreading the signal.

Epinephrine's actions are mediated through adrenergic receptors (sometimes referred to as adrenoceptors). It binds to receptors of liver cells, which activate inositol-phospholipid signaling pathway, signaling the phosphorylation of insulin, leading to reduced ability of insulin to bind to its receptors.

Epinephrine also activates adrenergic receptors of the liver and muscle cells, thereby activating the adenylate cyclase signaling pathway, which will in turn increase glycogenolysis. Receptors are found primarily in skeletal muscle blood vessels to trigger vasodilation. However alpha receptors are found in most smooth muscles and splanchnic vessels, and epinephrine triggers vasoconstriction in those vessels. Thus, depending on the patient, administration of epinephrine may raise or lower blood pressure, depending whether or not the net increase or decrease in peripheral resistance can balance the positive inotropic and chronotropic effects of epinephrine on the heart.

As a result of its vasoconstriction in topical use, epinephrine has a beneficial effect when combined in small quantities with lidocaine.

Sodium bicarbonate is a chemical compound with the formula NaHCO3 and its two-dimensional conformation is shown as follows:

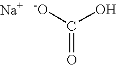

Because it has long been known and is widely used, the salt has many other names including sodium hydrogen carbonate and “sodium bicarb,” as well as baking soda, bread soda, saleratus, or bicarbonate of soda. It is poorly soluble in water. This white solid is crystalline but often appears as a fine powder. It has a slight alkaline taste resembling that of sodium carbonate. It is a component of the mineral natron and is found dissolved in many mineral springs. It is also produced artificially. As a result of its alkalinity in aqueous solution it plays a special role in the instant invention by its capacity to buffer the acidity of aqueous lidocaine (with or without epi).

NaHCO3 is mainly prepared by the Solvay process, which entails the reaction of sodium chloride, ammonia, and carbon dioxide in water. It is produced on the scale of 100,000 tons/year.

NaHCO3 also arises when sodium carbonate is treated with carbon dioxide. Commercial quantities of baking soda are produced by this method: soda ash, mined in the form of the ore trona, is dissolved in water and treated with carbon dioxide. Sodium bicarbonate precipitates as a solid from this solution:

Na2CO3+CO2+H2O=2 NaHCO3

NaHCO3 is a salt with a pH of 8.3 which consists of the ions Na+ and the bicarbonate anion, HCO3−. In aqueous solution, these ions are separated. Furthermore the bicarbonate anion forms some hydroxide, which results in its solutions being mildly alkaline. NaHCO3 is obtained by the reaction of carbon dioxide with sodium hydroxide.

Sodium bicarbonate is primarily used in cooking where it reacts with other components to release carbon dioxide, that helps dough “rise”. The acidic compounds that induce this reaction include cream of tartar, lemon juice, yoghurt, etc.) as a in baking: some forms of baking powder contain sodium bicarbonate combined with cream of tartar.

The reaction of acids with sodium bicarbonate is a common method for neutralizing acid spills. The advantage to this method is that one can use excess sodium carbonate, which is relatively innocuous. The neutralization process is signaled by the release of gaseous CO2. A wide variety of applications follow from its neutralization properties including ameliorating the effects of white phosphorus in incendiary bullets, from spreading inside a soldier's afflicted wounds.

Sodium bicarbonate is commonly used to increase the pH and total alkalinity of the water for pools and spas. Sodium bicarbonate can be added as a simple solution for restoring the pH balance of water that has a high level of chlorine, as in maintaining a healthy septic tank.

Sodium bicarbonate is also used as a deodorizer. An absorbent for moisture and odors, an open box can be left in a refrigerator for this purpose. Among its many uses include eliminating odors from vacuum flasks, as a substitute for talcum powder to prevent smelly feet, as a microwave cleaner, a hand cleaner, and to clean vegetables.

Sodium bicarbonate is also known for use as a pest control, as it kills fleas and drives away ants. It also has application in killing crab grass.

In medicine, sodium bicarbonate has been known as an admixture with vinegar into a paste salve for relieving burning from insect stings (particularly bee stings), poison ivy (to pull the poison out and dry up the ivy), nettles, and sunburn. It is also used as an antacid to treat acid indigestion and heartburn, and as a gargle to ameliorate flu, cold and throat symptoms.

Cosmetic uses of sodium bicarbonate include use in toothpaste, as baking soda helps to gently remove stains, whiten teeth, freshen breath, and dissolve plaque. Small quantities are placed in shampoo to remove residue from other products and increase hair luster. Washing the face with sodium bicarbonate cleans pores and prevents acne. Admixed with honey, sodium bicarbonate is also used for a face cleanser and moisturizer.

Industrially, a paste from baking soda has been shown to be effective when used in cleaning and scrubbing. Likewise, a warm aqueous solution removes tarnish from silver, and cleans impurities on contact lenses.

Sodium bicarbonate is considered to be relatively safe, however consumption of large amounts should be avoided.

Elsevier (Wong, DL) (see mosbysdrugconsult.com/WOW/op022.html) reports a proposed protocol comprising xylocaine (lidocaine) 1% (without epinephrine) buffered using a 10:1 ratio of lidocaine to sodium bicarbonate to be administered in an amount of 0.1 to 0.2 ml (buffered 20 ml vial of lidocaine 1% with 2 ml of sodium bicarbonate) into a 0.5 cc insulin wyrnige (with 28-30 gauge needle) to the side of a proposed entry of an intravenous catheter for venipuncture. Larson, et al. reports (ncbi.nlm.nih.gov/pubmed/2030202) upon use of buffered lidocaine for reduction of pain on injection. Lidocaine with epinephrine was buffered to a neutral pH to evaluate stability (shelf life). After storage for 4 weeks at 25° C., buffered lidocaine dropped to 66.1% of initial concentrations. Buffered epinephrine fell to 1.34% of its initial concentration under similar conditions. Both maintained 96.4% and 82.04%, respectively, of their initial concentrations after 4 weeks when refrigerated at 0-4° C., and greater than 90% concentration for both after 2 weeks of storage at that temperature. It was concluded that the shelf life was preserved for about 4 weeks when storage occurs at that temperature.

When stored at room temperature, it is reported that buffered lidocaine loses clinical activity when stored for more than 1 week at room temperature. (Hansen, D J, Good Samaritan Hospital)

The need to have lidocaine (with and without epinephrine) on hand for immediate use, while buffered at the same time to minimize the “stinging” effect, is apparent. Yet in order to maintain clinical efficacy, the composition needs to be refrigerated to avoid disposal, as a result of the issues of half life.

It is an object of the present invention to provide a method and apparatus for buffering lidocaine (with and without epinephrine) which provides improved shelf life without the need to refrigerate.

Further objects of the instant invention will be apparent upon reading and comprehension of the specifications, claims and figures, by one of ordinary skill in the art.

### SUMMARY OF THE INVENTION

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter in which there are illustrated and described preferred embodiments of the invention.

The foregoing objects and other objects of the invention are achieved through a method and apparatus for providing therapeutically effective mixtures of buffered lidocaine (with and without epinephrine) while simultaneously increasing the shelf life, having a two-chambered medicine vial, in which one chamber isolates generally 20 ml of a lidocaine solution (1% with or without epinephrine) and the other chamber isolates generally 2 ml of sodium bicarbonate (8.4%), in amounts that maintain the ratio of lidocaine to sodium bicarbonate of 10:1 (for optimal pH buffering. Between the chambers resides a fracturable wall such that the wall is fractured by bending or twisting, for mixing the lidocaine and sodium bicarbonate to create a buffered lidocaine for administration in therapeutically effective amounts, and further comprising stopper means, like a rubber stopper, for the insertion of a syringe for extraction of the mixture for administration.

It should be observed that the invention provides isolation of the components until usage is required, thereby eliminating the half life issue associated with mixing the two by hand prior to usage, and thereupon needing to throw the result away. In particular feature, where a surgeon needs immediate access to a therapeutically effective buffered lidocaine to minimize pain associated with unbuffered lidocaine, the method and apparatus create a simple ability to grab the apparatus, break the connection between the two chambers, and shake and administer the resultant composition. No longer must that surgeon reach for two different vials, mix the two by hand, and use the result. No longer will a buffered lidocaine that has been previously created, result in a concern over efficacy without knowing when the mixing had occurred, since it is known that shelf life is but a week without refrigeration, and about 4 weeks with refrigeration. Now featured, a surgeon can mix upon demand in simple fashion.

Other features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings, wherein similar reference characters denote similar elements through the several views:

FIG. 1 is an overview of the method and system for buffering lidocaine, in accordance with a preferred embodiment of the subject invention;

FIG. 2 shows a cross-sectional representation of an apparatus for buffering lidocaine for administration, in accordance with a preferred embodiment of the subject invention;

FIG. 3 shows a cross-sectional representation of another apparatus for buffering lidocaine for administration, in accordance with an additional preferred embodiment of the subject invention; and

FIG. 4 shows a cross-sectional representation of an additional apparatus for buffering lidocaine for administration, in accordance with a still additional preferred embodiment of the subject invention.

### DETAILED DESCRIPTION OF THE PREFERRED EMBODIMENTS

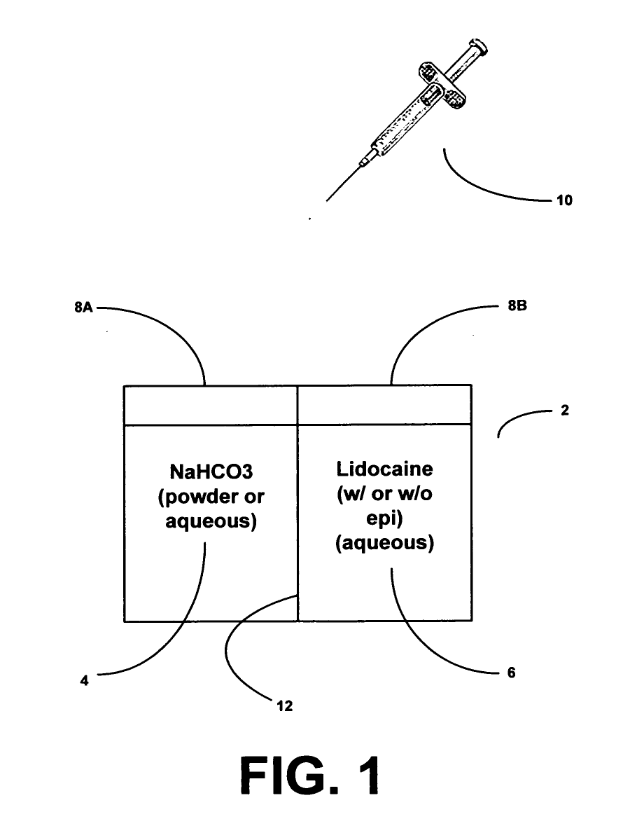

In accordance with the subject invention, FIG. 1 is a general overview of the preferred method and apparatus of the subject invention, wherein vial 2 comprises an isolation between lidocaine (with or without “epi,” a common abbreviation for epinephrine) in chamber 6 and sodium bicarbonate (either in powder or aqueous form) in chamber 4. Between chambers 4 and 6 is fracturable wall 12. It should be appreciated that while on the shelf, the isolation between chambers 4 and 6 improves shelf life, as once the chambers are mixed therapeutic degradation results over time.

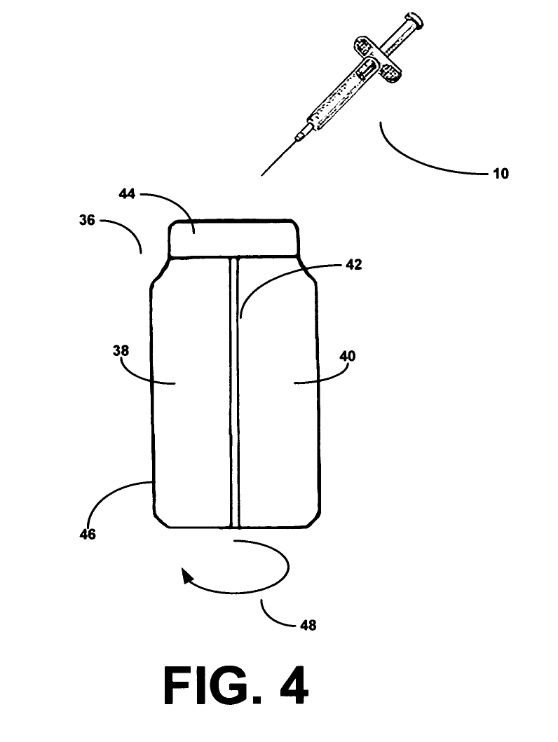

Vial 2 has stopper means 8A and 8B, which is generally a rubber stopper that can be pierced by syringe 10 for the purpose of extracting liquid for administration. Stopper means 8A and 8B are shown in this embodiment as having two components (A and B) as wall 12 can be fractured by twisting, in at least one embodiment, shown in greater detail in FIG. 4.

For administration, fracturable wall 12 is fractured by twisting or bending, as described in greater detail hereinbelow, such that the lidocaine in chamber 6 and sodium bicarbonate in chamber 4 can be mixed together for neutralizing the pH. Mixture occurs typically by shaking vial 2, after the components in chambers 4 and 6 are mixed, and prior to extraction by syringe 10 through stopper means 8A/8B.

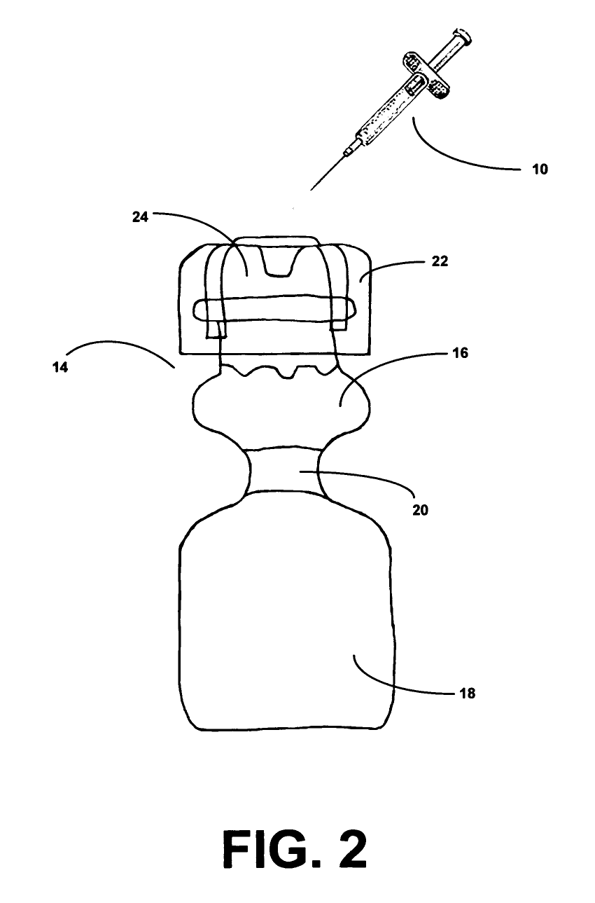

FIG. 2 is a cross-sectional representation of a dual chambered apparatus 14, wherein each of lidocaine and sodium bicarbonate are isolated in chambers 16 and 18, separated by collar 20. In use, cap 22 is separated from upper portion 24 of apparatus 14 (generally by turning to break the seal and removal), whereupon syringe 10 is utilized to pierce stopper 20 allowing fluid in chamber 16 to mix with the contents of chamber 18. Thereupon the mixture can be shaken to achieve full neutralization, and syringe 10 is used to pass through collar 20 and extract the mixed, buffered/neutralized composition from chamber 18, for administration.

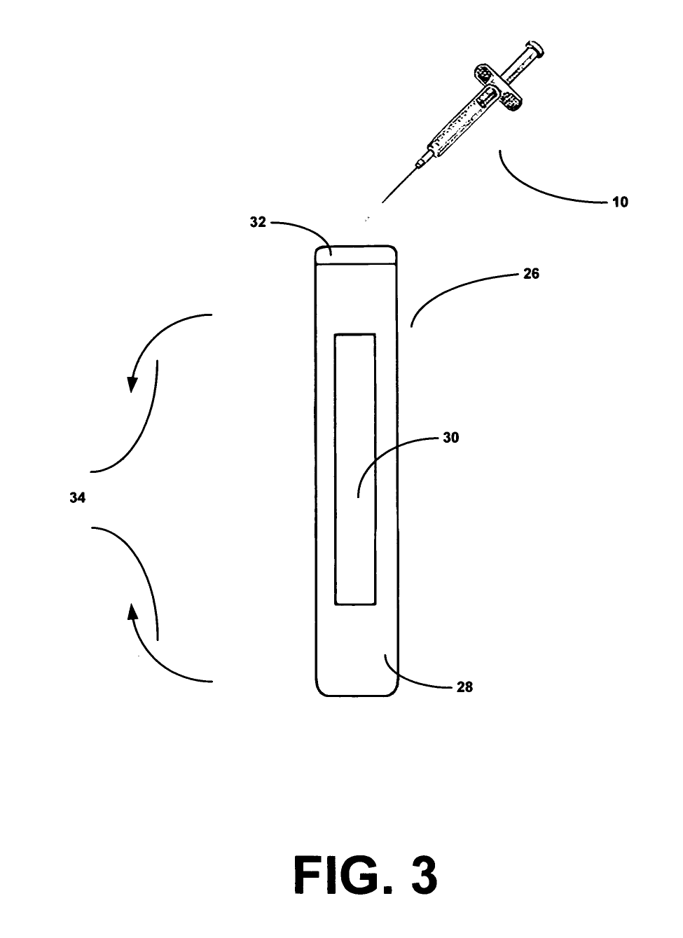

FIG. 3 is a cross-sectional representation of an alternative, preferred embodiment of the subject invention, in which vial 26 has a dual chambered configuration in which an internal chamber 30 is presented in an external chamber 28. In this embodiment, vial 26 actually comprises the second external chamber 28. In accordance with arrows 34, vial 26 is bendably engaged such that internal chamber 30 is caused to fracture, releasing its contents to external chamber 28. In this embodiment, chamber 30 is typically comprised of a fracturable material like glass, and external chamber 28 of a deformable material, like a bendable plastic. In this manner while internal chamber 30 is caused to fracture when vial 26 is bendably engaged along lines 34, external chamber 28 does not release its contents. Once chamber 30 is fractured, the solution is mixed for neutralization, and syringe 10 is engaged to extract fluid via stopper means 32. It should be appreciated that stopper means 32 (like 8A/8B and 20) provides a permeable barrier for extraction with syringe 10, while preventing leakage to the outside. Typical rubber vial stopper means, known to those of ordinary skill in the art, are utilized for this portion of the apparatus.

FIG. 4 shows an alternative, preferred embodiment of vial 36, wherein stopper means 44 attaches to fracturable wall 42 such that turning stopper means 44 in accordance with arrow 48 causes the attached wall 42 to twist and fracture, thereby releasing the composition from each of chambers 38 and 40 in containment 46 to mix for extraction via syringe 10, after mixing and neutralization is complete.

It should be appreciated that the method of administrations is also herein provided, in that a therapeutic amount of buffered lidocaine is provided by isolating a therapeutic amount of lidocaine in solution and a pH-neutralizing amount of buffering reagent in a single medicine vial having a breachable wall between said amounts of lidocaine and buffering reagent; breaching said wall; mixing said lidocaine and said buffering reagent; and withdrawing a suitable quantity of said mixture for administration.

In these embodiments, said lidocaine solution comprises 1% lidocaine, with or without epinephrine. A buffering reagent is provided, preferably sodium bicarbonate, either in granulated powder form or 8.4% in solution. The amounts isolated are in ratio of 10:1, lidocaine to sodium bicarbonate, and preferably 20 ml of 1% lidocaine and 2 ml. of sodium bicarbonate in 8.4% solution. Ten-to-one dilutions of lidocaine are prepared by replacing 2 mL of 1 percent lidocaine from 20-mL multidose vials with 2 mL of diluent. Diluents are sodium bicarbonate (44 mEq per 50 mL).

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

I claim:

1. An apparatus for extending the shelf life of buffered lidocaine for administration in therapeutic amounts, comprising a two-chambered medicine vial, wherein one chamber isolates a lidocaine solution and the other sodium bicarbonate with a fracturable wall therebetween such that the wall is fractured for mixing said lidocaine solution and sodium bicarbonate to create a buffered lidocaine for administration, and further comprising stopper means for the insertion of a syringe for extraction of said mixture for administration.

2. The apparatus of claim 1, wherein said lidocaine solution comprises 1% lidocaine.

3. The apparatus of claim 1, wherein said lidocaine solution further comprises epinephrine.

4. The apparatus of claim 1, wherein said sodium bicarbonate is a granulated powder.

5. The apparatus of claim 1, wherein said sodium bicarbonate is 8.4% in solution.

6. The apparatus of claim 1, wherein said apparatus is of sufficient size to accommodate the mixture of lidocaine to sodium bicarbonate in a ratio of 10:1.

7. The apparatus of claim 1, wherein said apparatus is of sufficient size to accommodate the mixture of 2 ml of said sodium bicarbonate in 8.4% solution to 20 ml of 1% lidocaine.

8. The apparatus of claim 1, wherein said wall is breached by twisting.

9. The apparatus of claim 1, wherein said wall is breached by bending.

10. A method for administering a therapeutic amount of buffered lidocaine, comprising:
(a) isolating a therapeutic amount of lidocaine in solution and a pH-neutralizing amount of buffering reagent in a single medicine vial having a breachable wall between said amounts of lidocaine and buffering reagent;
(b) breaching said wall;
(c) mixing said lidocaine and said buffering reagent; and
(d) withdrawing a suitable quantity of said mixture for administration.

11. The method apparatus of claim 10, wherein said lidocaine solution comprises 1% lidocaine.

12. The method of claim 10, wherein said lidocaine solution further comprises epinephrine.

13. The method of claim 10, wherein said buffering reagent is sodium bicarbonate.

14. The method of claim 13, wherein said sodium bicarbonate is a granulated powder.

15. The method of claim 13, wherein said sodium bicarbonate is 8.4% in solution.

16. The method of claim 13, wherein the amounts isolated are in ratio of 10:1, lidocaine to sodium bicarbonate, and preferably 20 ml of 1% lidocaine and 2 ml. of sodium bicarbonate in 8.4% solution.

17. The method of claim 10, wherein said breaching occurs by twisting.

18. The method of claim 10, wherein said breaching occurs by bending.
